(19) United States
(12) Patent Application Publication
(10) Pub. No.: US 2024/0189058 A1
(43) Pub. Date: Jun. 13, 2024

(54) BREATH SHIELD

(71) Applicants: Kathryn Najafi-Tagol, Tiburon, CA (US); Sara Tagol, Tiburon, CA (US); Ramin Najafi, Tiburon, CA (US)

(72) Inventors: Kathryn Najafi-Tagol, Tiburon, CA (US); Sara Tagol, Tiburon, CA (US); Ramin Najafi, Tiburon, CA (US)

(21) Appl. No.: 18/556,621

(22) PCT Filed: Apr. 20, 2022
(86) PCT No.: PCT/US2022/025618

§ 371 Date: Oct. 20, 2023

Related U.S. Application Data
(60) Provisional application No. 63/177,030, filed on Apr. 20, 2021.

(51) Int. Cl. A61B 90/00; A61B 3/135; A61B 90/40

(52) U.S. Cl. CPC A61B 90/05; A61B 3/135; A61B 2090/0807; A61B 2090/401

(57) ABSTRACT

In some implementations, disclosed is/are a device, system and/or method for pathogen deflection including a) a shield member; and, one or both: b) a filtration sub-system; and/or c) a pressure differential subsystem. The filtration and/or pressure differential sub-assemblies may be or may include: positive or negative pressure differential or both and/or filtration sub-assemblies which may be: as pressure differential devices, these may include one or more fans or one or more mini-fans; and, as filtration devices, these may include HEPA or charcoal other filtration arrangements.

### BACKGROUND

In some implementations, the present developments relate to breath shields and may include filtration and/or pressure differential for more protection. In many implementations, devices hereof may be used to shield pathogens and/or filter pathogens and/or use pressure differential to reduce infiltration of airborne pathogens, or airborne particles, gas, liquid.

The COVID-19 pandemic has shown the importance of infection control. For some non-limiting examples, a face-to-face proximity of clinicians and patients during a slit lamp examination places ophthalmologists and optometrists at a high risk of exposure to aerosolized particles from respiratory droplets and the patient's breath. Similarly, patients may be at risk from an asymptomatic infected clinician examining them.

There are numerous breath shield devices known. However, desiderata remain for one or more of improved air deflection and/or functionality in pathogen reduction, particularly for airborne pathogens. The presently disclosed developments may be implemented to meet these and/or other desiderata.

### SUMMARY

Disclosed are a variety of breath shields that include a main shield portion and either or both a filtration and/or air pressurization, often positive pressure, sub-system.

A device hereof may include a shield structure or device for airborne shielding which in some implementations may include a Carbon and/or HEPA filter sub-system and/or a mini-fan sub-system.

### BRIEF DESCRIPTION OF THE DRAWINGS

FIG. 1 is a perspective view of an implementation of a device and some details hereof incorporating some one or more features of the present developments.

FIG. 2 is a perspective view of a device hereof, showing various components of a device hereof, as configured for use.

FIG. 3 is an enlarged depiction of a device as shown in FIG. 2.

FIG. 4 is a further enlarged portion of a device as shown in FIGS. 2 and 3.

FIG. 5 is an isometric view of a device hereof.

FIG. 6 is an isometric view of a portion of a device hereof.

FIG. 7 is an elevational view of a portion of a device hereof during manufacture.

FIG. 8 provides plan and elevational views of a portion hereof.

FIG. 9A provides plan and elevational and isometric views of a further portion hereof.

FIG. 9B provides an elevational view of a further portion hereof.

FIG. 10 provides plan and isometric views of a further portion hereof.

FIG. 11 provides plan and elevational and isometric views of a further portion hereof.

FIG. 12 provides plan and isometric views of a further portion hereof.

FIG. 13 provides plan and elevational and isometric views of a further portion hereof.

FIG. 14 is a perspective view of an alternative implementation of a device hereof.

FIG. 15 is a perspective view of an alternative implementation of a device hereof.

FIG. 16 provides an exemplary computer system or computing resources of a device hereof.

FIG. 17A provides a methodology for operating a device hereof.

FIG. 17B depicts an alternative methodology for operating a device hereof.

FIG. 18 depicts yet another methodology for operating a device hereof.

### DETAILED DESCRIPTION

While there has been described what is presently considered to be certain implementations of the developments, it will be understood that various modifications may be made therein, and it is intended to cover in the appended claims all such modifications as fall within the spirit and scope of the developments. The intention is to cover all modifications, equivalents, and alternatives falling with the spirit and scope of the developments whether described here or otherwise being sufficiently appreciable as included here within even if beyond the literal words hereof.

Effective control of the spread of infection may include reducing the risk of transmission by using enhanced barrier systems in addition to wearing masks and practicing proper hand hygiene.

Selection and utilization of respiratory personal protective equipment have become a concern in the COVID-19 pandemic. At least 50% of new SARS-CoV-2 infections are estimated to have originated from exposure to infected individuals who were asymptomatic. (JAMA Jan. 7, 2021 Johansson, Michael).

As described and shown herein, air pressure differentials to induce air flow, typically positive air pressure, and/or filtered barriers, in some cases, carbon filtered and/or HEPA (high-efficiency particulate air) filtered barriers and/or breath shields may exhibit an advantage of a reusable physical barrier as well as a HEPA filtered air pressure barrier in settings where a helmet cannot be worn such as in front of a slit lamp microscope or any other situation where a physical barrier can be placed to separate people's interactions.

Pressure differential, e.g., often a positive air pressure and/or HEPA (high-efficiency particulate air) filtered barriers and/or breath shields may provide as shown herein a reusable physical barrier as well as HEPA filtered air pressure barrier in settings to separate people's interactions. Other filter options could be used in addition and/or instead.

Some devices hereof may reduce the risk of infection by maintaining a pressure differential, e.g., positive pressure, and/or filtered air which often flows vertically and uniformly from above in front of the shield at a velocity sufficient to remove any infective particles in suspension within seconds. Choice of dimensions and/or a curved barrier or side panels may also help prevent exposure of respiratory droplets while still allowing access to the patient sitting behind the slit lamp or other person to person disposition.

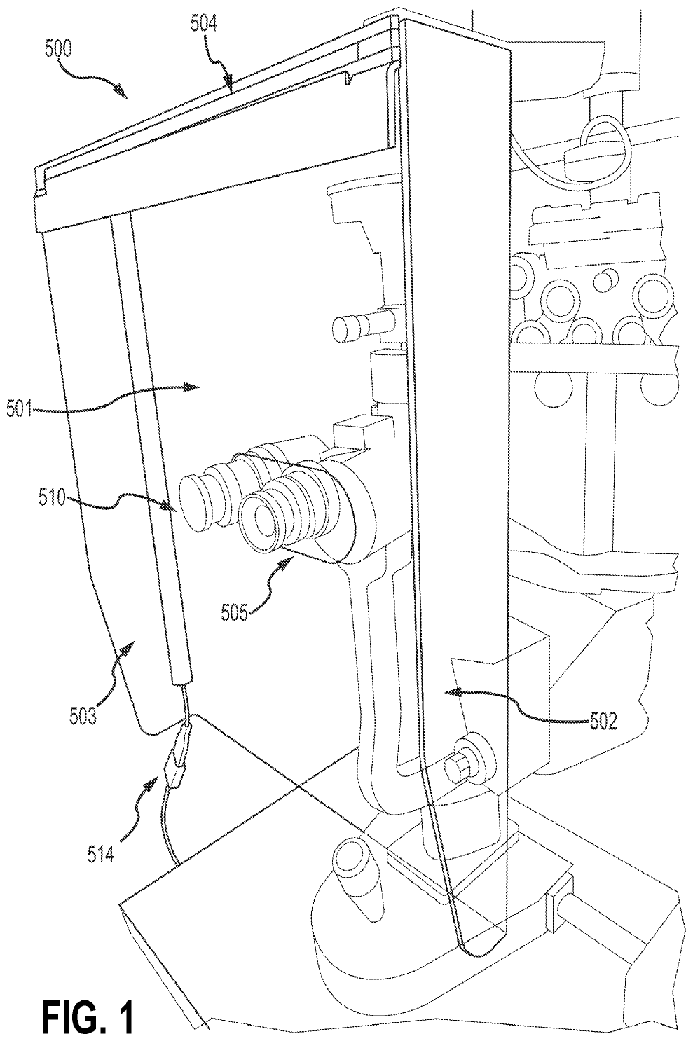

In a first implementation, the present developments relate to a breath shield device 500, as shown in FIG. 1, that may in many cases be optionally attachable or attached to other equipment such as ophthalmology slit lamp or like devices or the like.

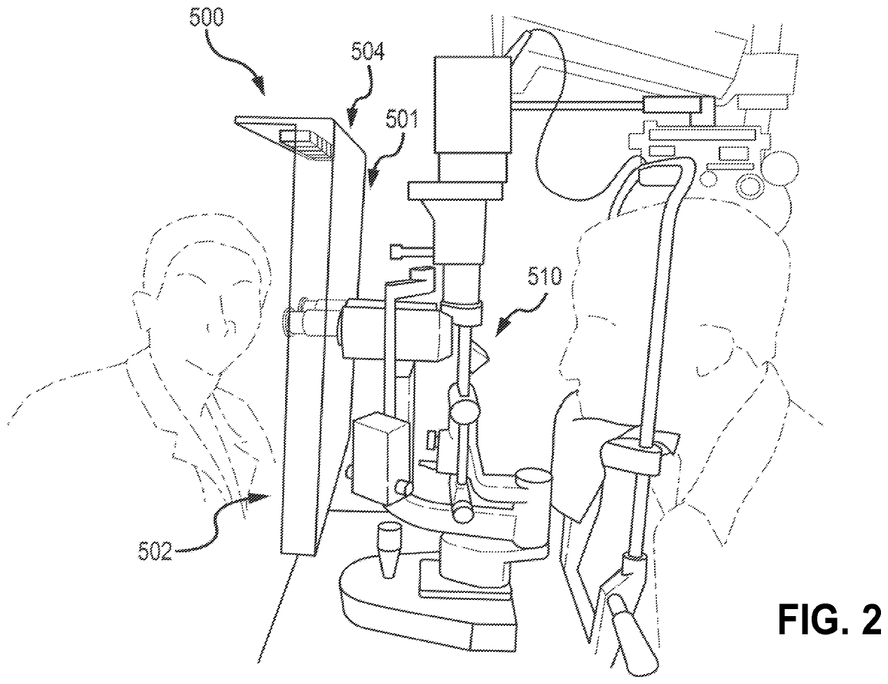

Such a device 500 is in this first implementation (as shown and described relative to FIGS. 1-3, inter alia), shown attached to a conventional slit lamp ophthalmology device 510.

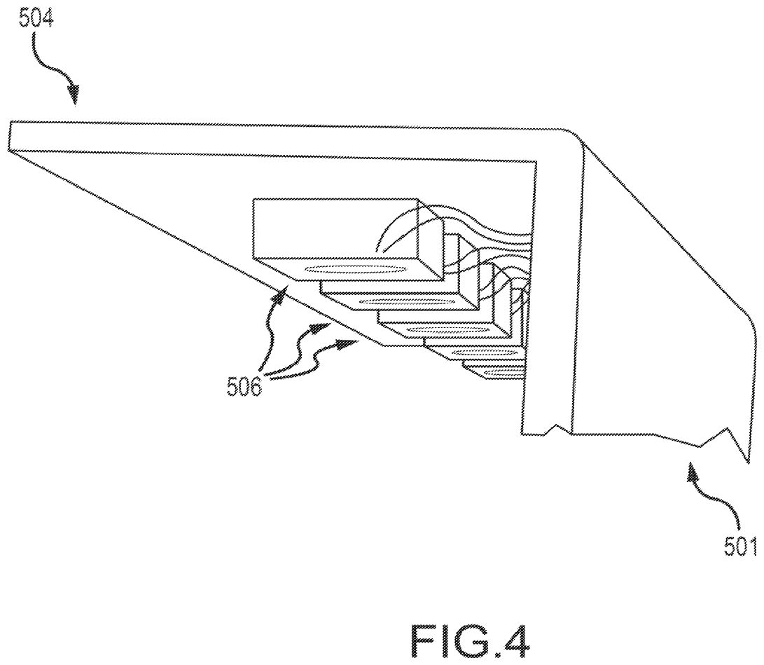

The first implementation 500 has a primary shield portion 501, an optional first side portion 502, a second optional side portion 503 and an optional top portion 504. Also shown in this implementation is an optional slot or aperture 505 for reception of a portion of a slit lamp device 510 or like device. FIG. 1 also depicts a power cord 514 that may be used to provide power to the device, including one or more sub-assemblies of FIG. 4, inter alia.

FIG. 4 shows in greater enlargement a further portion of a device 500, including the top portion 504 adjacent the main front portion 501. Also, shown are several sub-assemblies 506 which here are positive pressure and/or filtration sub-assemblies. As pressure differential devices, these sub-assemblies 506 include one or more fans or one or more mini-fans within each sub-assembly 506; and, as filtration devices, these include HEPA or other filtration arrangements; the filter being above or below the fan in the respective sub-assembly 506. The number and/or size of fans and/or sub-assemblies 506 can be varied relative to the practical application, personal usage implicating fewer and/or smaller fans, larger scale operations indicating larger or more fans for/during operation. Only practicality limits how many or how large; whether no fans, one fan, a few fans, or a larger number of plural fans may be implemented. For the filters, though choice may allow for alternative filters; HEPA filter(s) may be used, and may be particularly applicable for bacteria and viruses and other filters might be used particularly for other desiderata; e.g., Carbon filter(s) may be used to reduce chemical odors such as odor or cosmetics, perfume, etc.; electrostatic filters or polarized media electronic air filters may also be used in some instances.

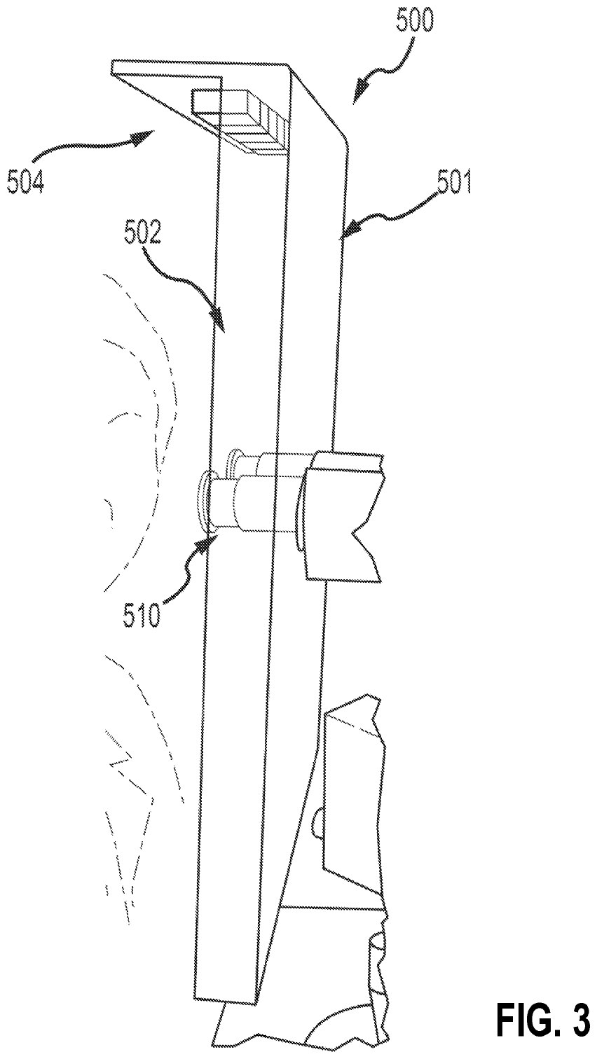

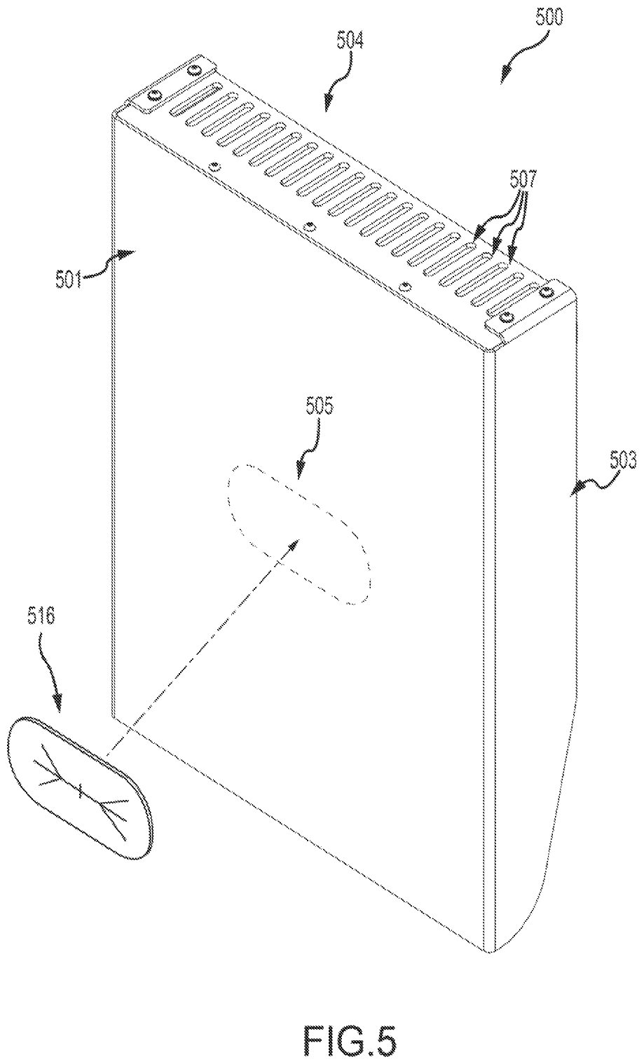

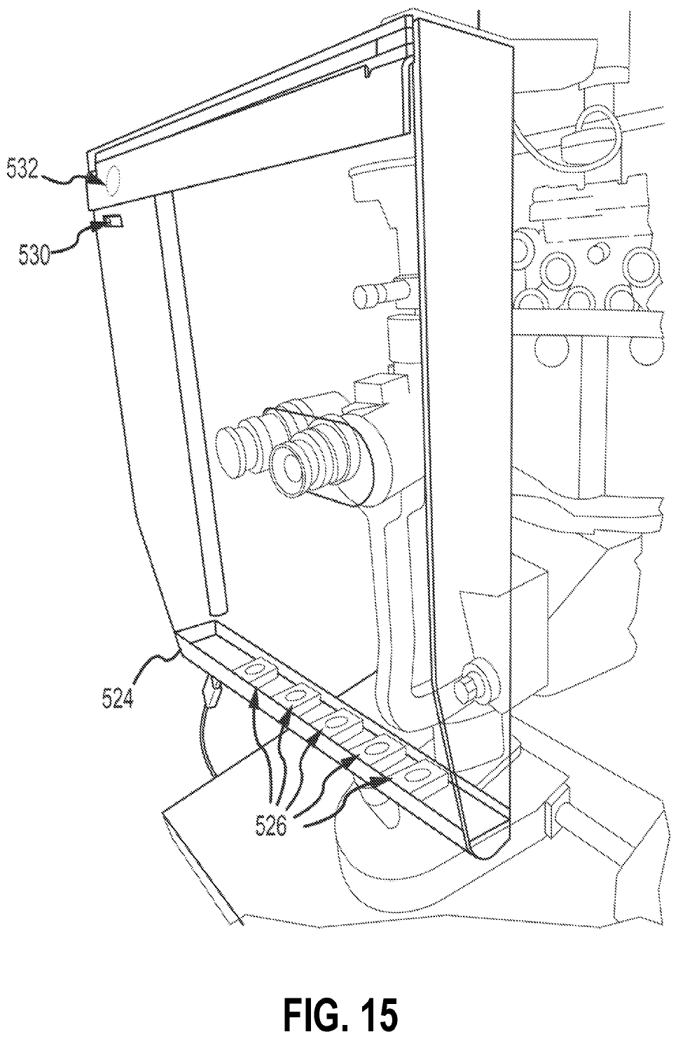

FIG. 5 provides an alternative view of a device 500 including the main front or primary shield portion 501, one of the optional side portions 503 and the top portion 504. Integrated together with the version of FIGS. 3 and 4, inter alia, the top portion 504 could/would have included the optional sub-assembly or sub-assemblies 506 (not directly shown in FIG. 5, and/or 526, see FIG. 15, e.g.). A grill or set of apertures 507 is shown as part of the top portion 504 to indicate air inlets for the pressure differential and/or filtration sub-assemblies. FIG. 5 also shows the aperture 505 in dashed lines to indicate the optionality hereof. In some implementations, a gasket 516, or baffle, made of rubber, vinyl, latex, plastic or other partially deformable material may be placed in aperture 505 to further seal the aperture; however, the gasket 516 may have, as shown, one or more slits to allow the ophthalmologic device to extend through the aperture 505.

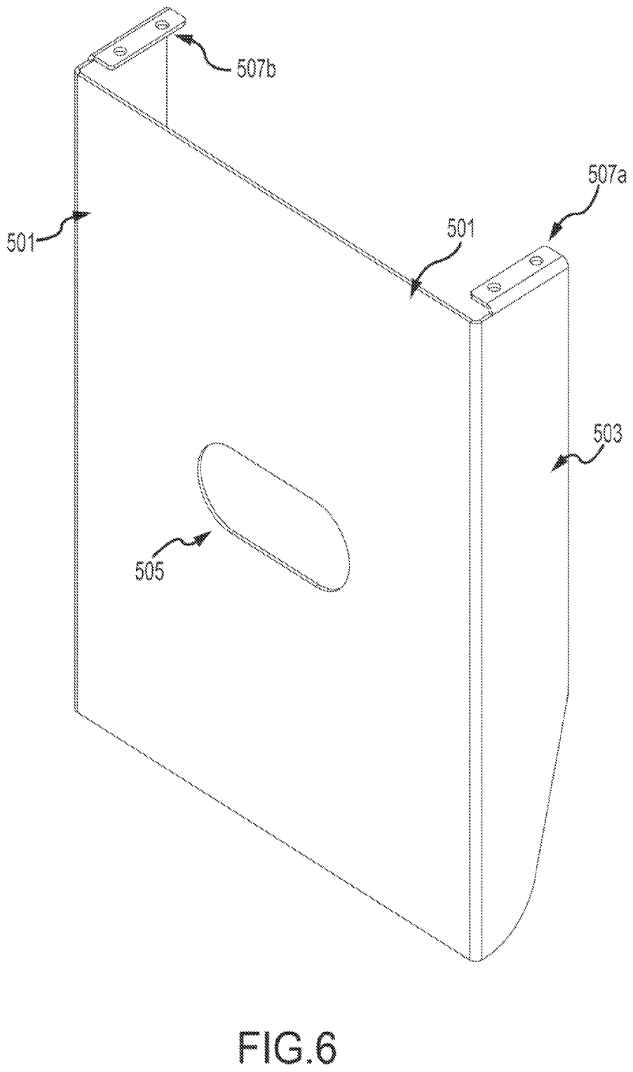

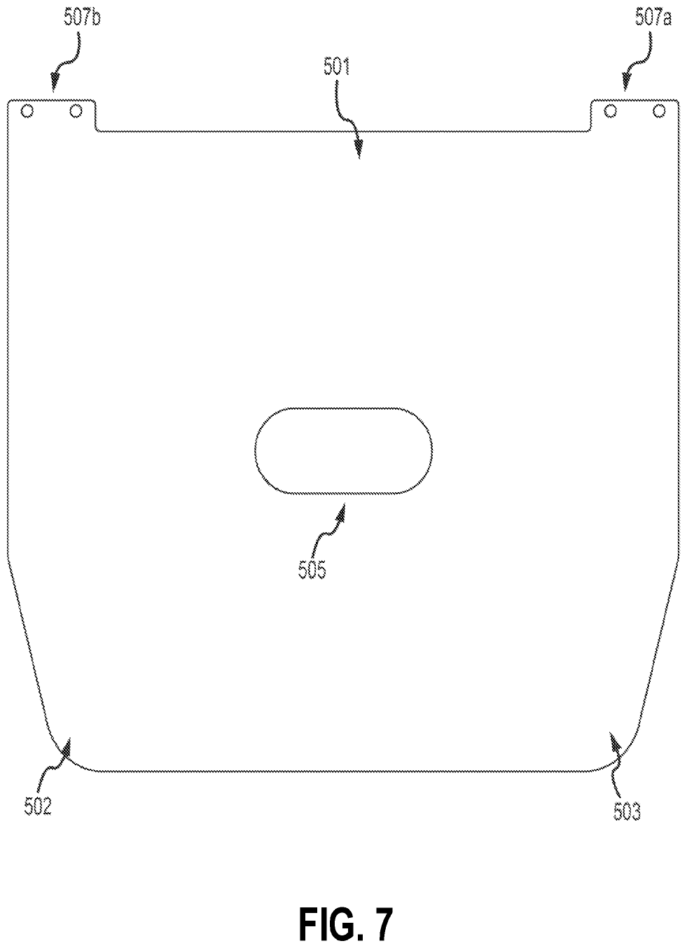

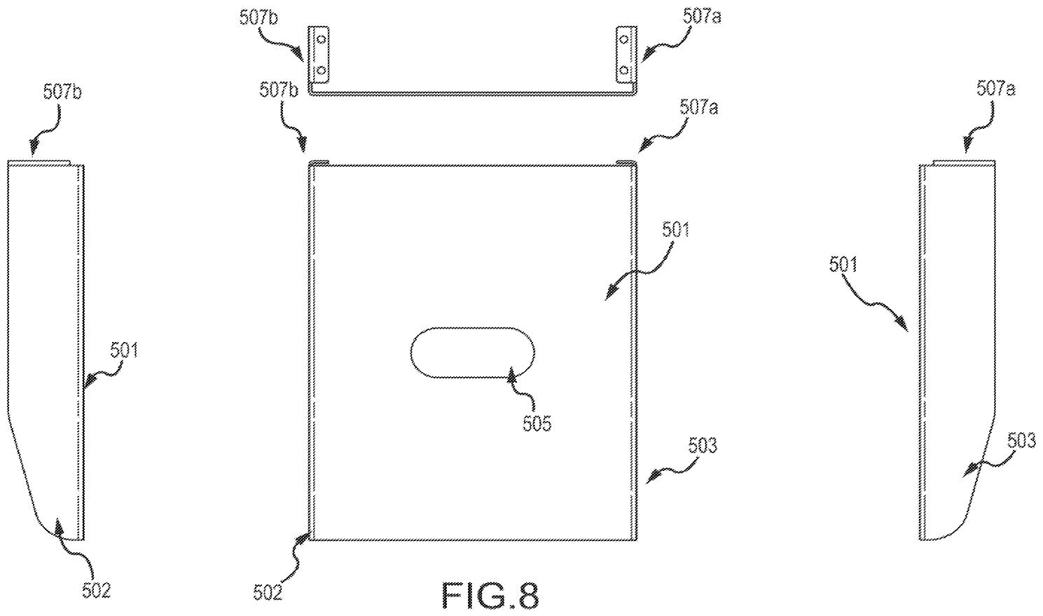

FIG. 6 is not unlike FIG. 5 though without the top portion 504. Rather here shown is a set of relative attachments 507a and 507b to which a top portion 504 and/or grill 507 might be attached. FIG. 7 is another view, here elevational, of the front portion 501 with both side portions 502 and 503 and the attachments 507a and 507b. In this view, it may be seen that a shield portion may be cut, molded or otherwise formed as a relatively single part that may initially be flat and then bent to take the forms shown in FIGS. 1-6. The optional aperture 505 is shown here as well. FIG. 8 shows elevational, side and plan views of the primary shield member 501 with the respective sides 502, 503, aperture 505 and the attachments 507a and 507b.

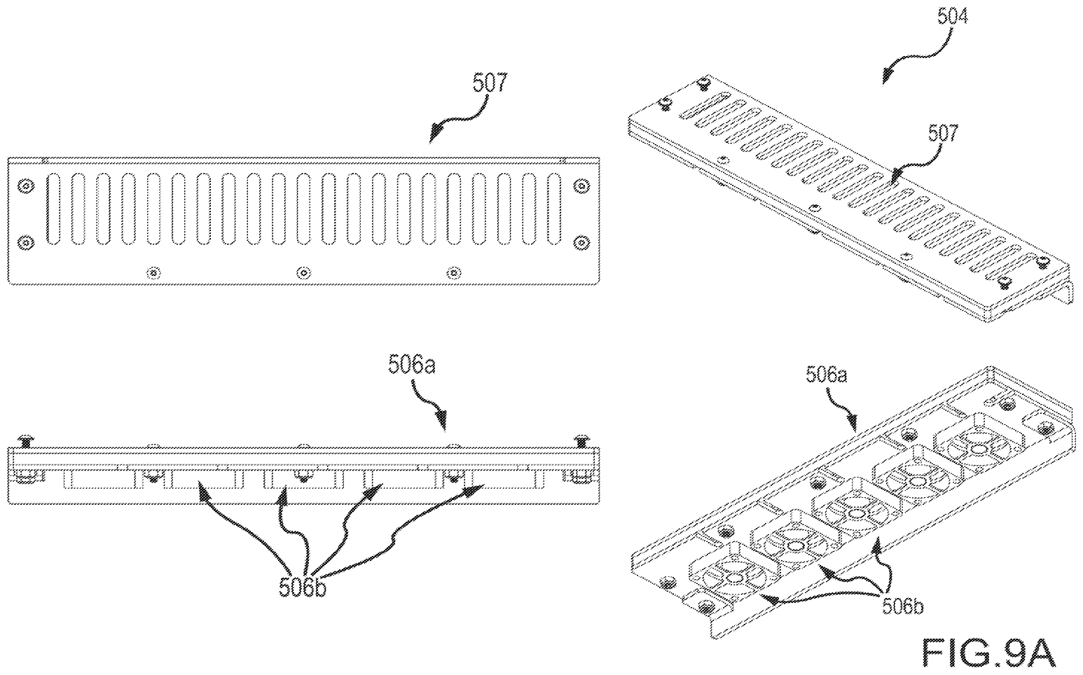

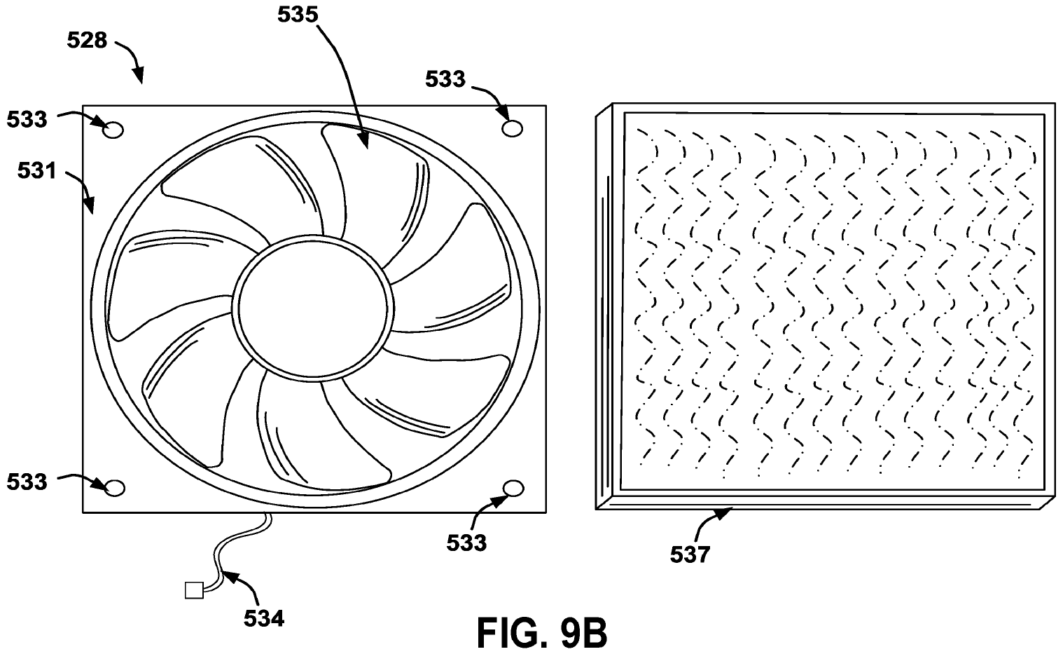

FIG. 9A shows the top member 504 not attached to the primary shield member of FIGS. 6-8, inter alia. A grill member 507 is shown detached from a sub-assembly holder 506a that includes several of one or more filtration and/or pressure differential sub-assemblies 506b.

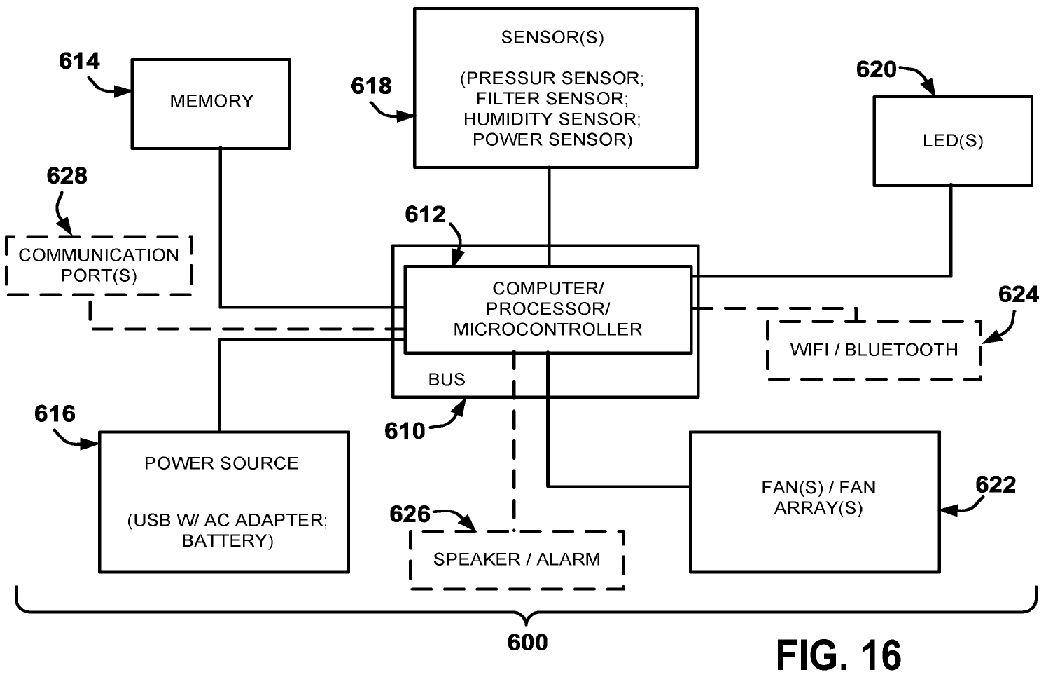

FIG. 9B shows a fan 528 that may be inserted and cooperatively connected to power and placed inside or otherwise integrated with the sub-assembly. The fan 528 may have a mounting structure 531, one or more mounting holes 533 to allow the mounting structure 531 to be attached and/or secured within the sub-assembly via a screw (not shown). The fan 528 may have one or several blades 535, as further shown in FIG. 9B. The fan 528 may also have a power cord/connection 534 that allows the fan to be cooperatively connected to a power source and/or control electronics, described in FIG. 16, inter alia. Also shown in FIG. 9B is a filter 537 that is intended to be placed or layered on top of or underneath the fan 528 to provide the filtration, purification, and/or air processing as described elsewhere herein.

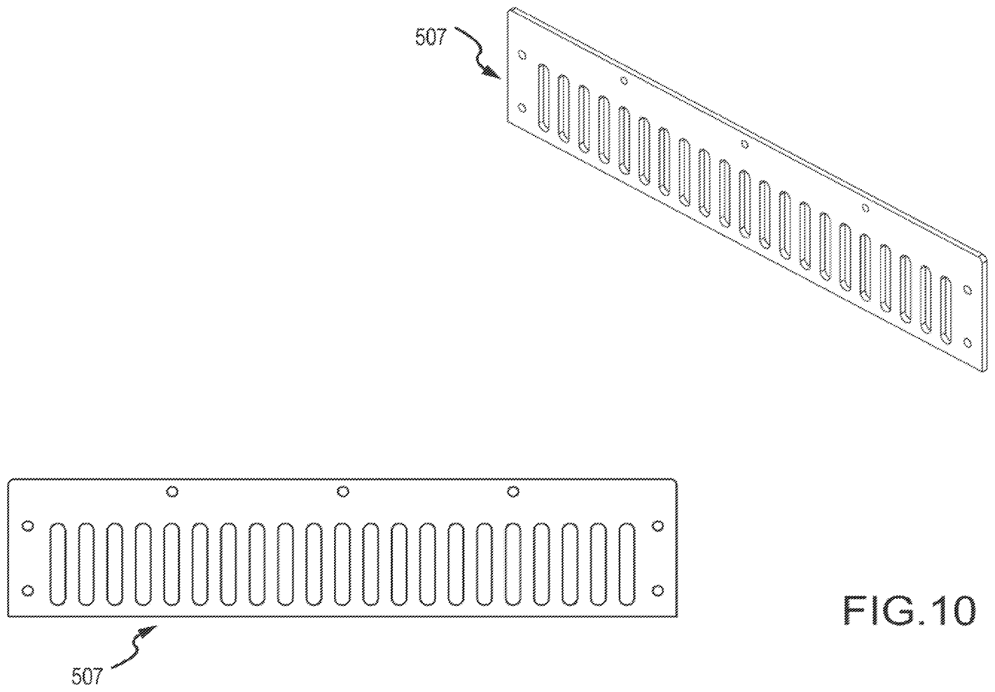

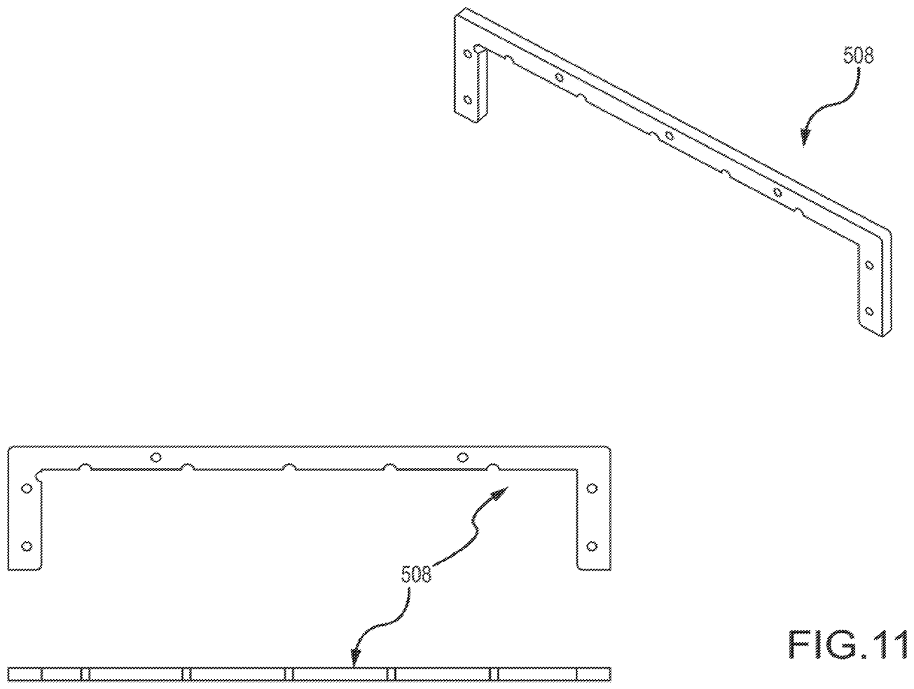

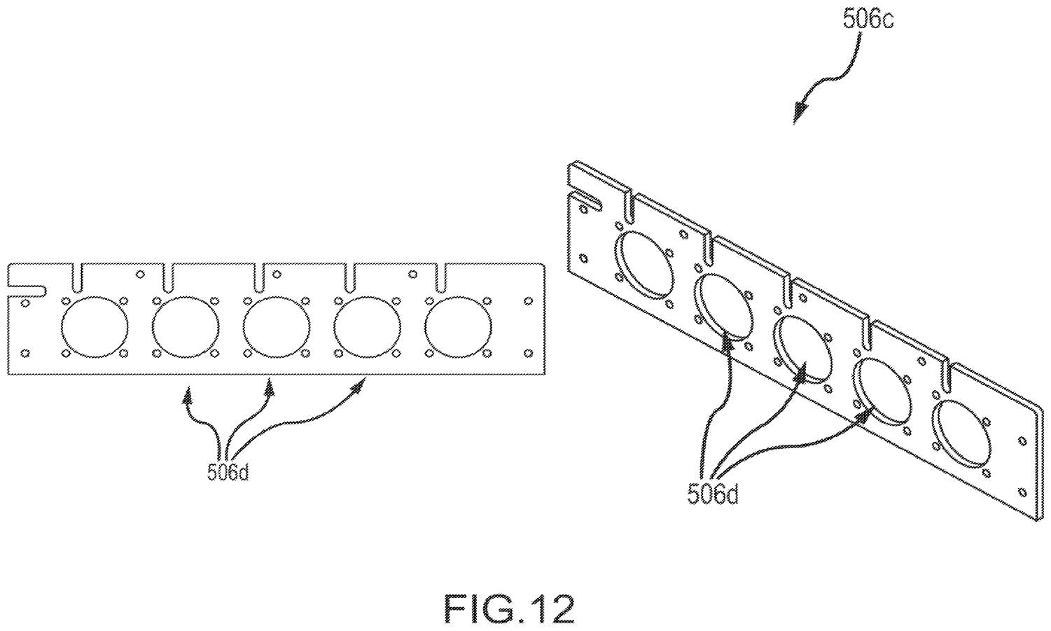

A grill 507 is shown separately in FIG. 10. A spacer 508 for use in the top member 504 is shown in FIG. 11. A further spacer 506c is shown in FIG. 12 with apertures 506d to assist in defining the filtration and/or positive pressure sub-assembly holders.

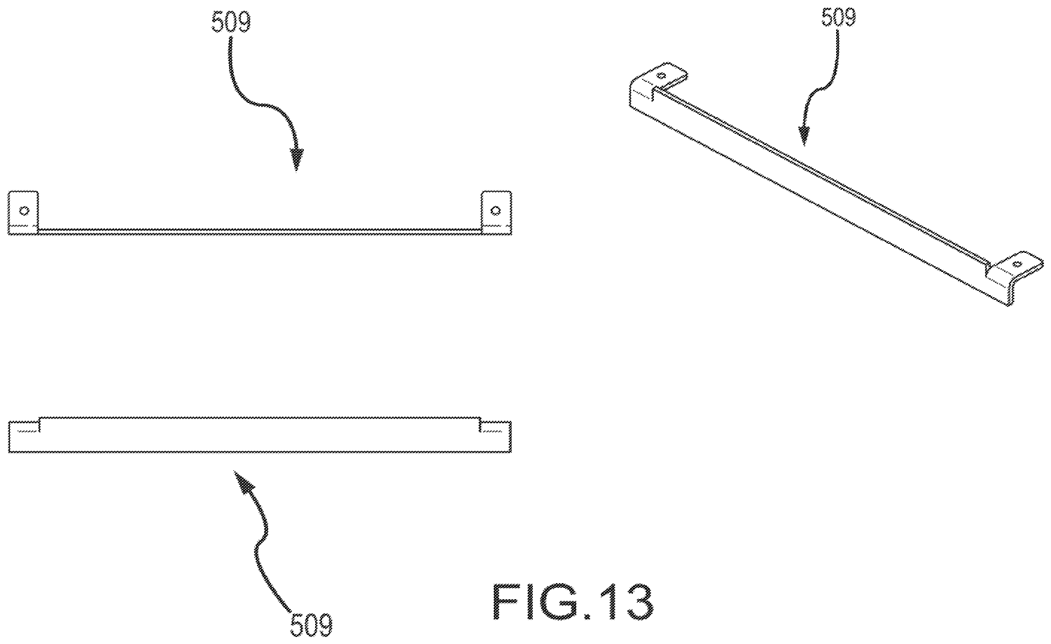

A lip member 509 is shown in FIG. 13 which may be attached to and/or form a part of the top member 504 to assist in directing positive pressure flow and/or further assist in deflection.

The combination of elements described can thus form a fanned positive pressure slit lamp doctor-patient protection device 500. In some examples, the device 500 can be used to deflect pathogens, as for example experienced in the current COVID-19 pandemic which has shown the importance of infection control. The face-to-face proximity of clinicians and patients during a slit lamp examination or like person to person interactions places ophthalmologists and optometrists or other service providers at a high risk of exposure to aerosolized particles from respiratory droplets and the patient's breath. Similarly, patients may be at risk from an asymptomatic infected clinician examining them.

In one implementation, the protection device 500 may be positioned and held in place on the slit lamp device via a bottom stand that provides adequate support to keep the device substantially upright and vertical. In another implementation, the protection device 500 may be positioned and held in place by one or more self-tapping eye hooks or ring hooks that are attached to the top portion and allow the device to be attached to the ceiling via a lightweight chain or picture hanging wire, or the like. In another implementation, the device may be positioned, rested, and supported by slit lamp as it extends through the aperture 505.

The options herein described may include either or both enhanced positive (or negative) air pressure and/or filtration barriers, as e.g., HEPA (high-efficiency particulate air) filtered barrier with a breath shield to provide an advantage of a reusable physical barrier as well as HEPA filtered air pressure barrier in settings where a physical barrier can be placed to separate people's interactions.

A device 500 may reduce the risk of infection by maintaining a positive pressure (or pressure differential) filtered air which flows vertically and uniformly from above in the airspace between the shield and the user of the shield at a velocity or flow rate sufficient to remove any infective particles in suspension within seconds. Larger dimensions and/or a curved barrier may also help prevent exposure of respiratory droplets while still allowing access to the patient sitting behind the slit lamp. HEPA filtration can be helpful particularly with infective particles, e.g. bacteria, viruses or other pathogens.

Positive pressure air flow with filtration may be provided by one or more or in many example cases ideally by 3-5 fans equipped with 1-3 layers of HEPA filters situated above a shield system to separate the air flow of patient and doctor or any two individuals (non-limiting examples). Less optimum but possibly viable may be use of either positive pressure or HEPA filtration separately from each other. Other implementations may include alternative fan or filtration dispositions other than at the top; as e.g., these may alternatively and or additionally be on one or both sides, or even at the bottom, though may be preferred with the one or more shield minifans at the top and charcoal filter or HEPA filter flushing down the face.

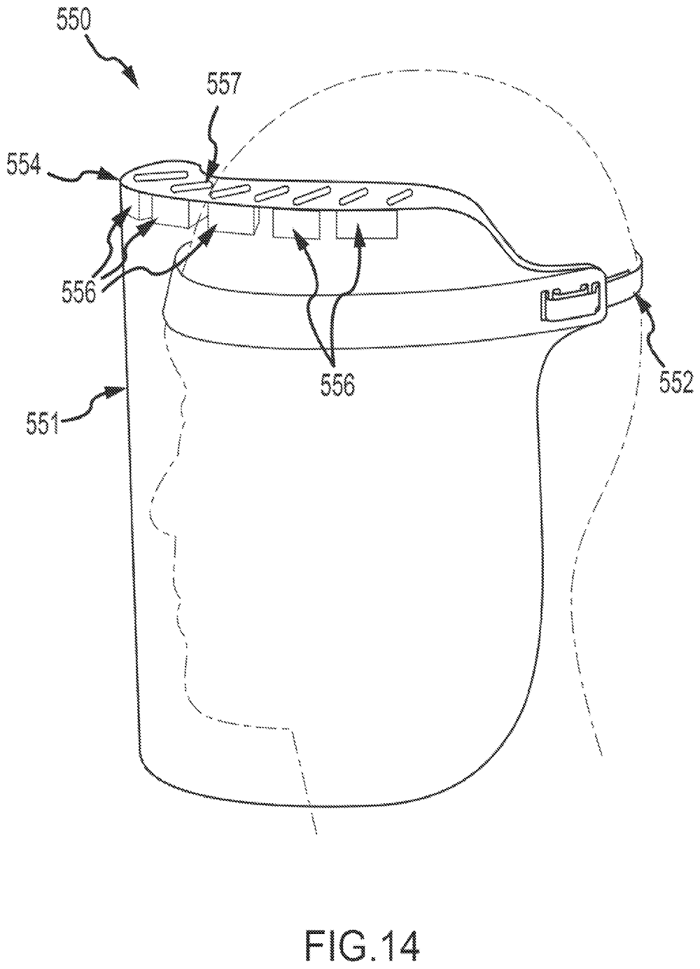

In other embodiments of such device; a face shield with 2-3, or more, mini fans on the top, may be attached to a rechargeable battery either mounted on the device itself or elsewhere on the user as for example, mounted to a belt. An example of such a device is further described and depicted in FIG. 14, described in more detail below.

The present device 500 improves on a basic acrylic shield found in many stores by offering filtered air, e.g., HEPA filtered, or using one or more mini fans.

The present device has a shield that provides protection for both patient and doctor while also providing either or both filtration and/or pressure differential e.g., positive pressure, as e.g., HEPA filtered air and one or more fans to prevent aerosolized infectious particles from reaching either person.

Moreover, face shields have previously been provided that many people use for individual use. However, the present device has also provides filtered air (e.g., HEPA filtered) and/or along with one or more pressure differential devices or fans to induce air flow in the user air space to add to the level of protection that a smaller face shield would have.

In other embodiments, other person-to-person interactions can make use of devices 500 hereof. E.g., a banker can use the present device for protection from an infected customer. In another embodiment a nail salon operator can use it to protect against an infected customer. In another embodiment, a merchant can protect themselves from another customer. In yet another embodiment, a carbon filter can be used instead and/or added to a HEPA filter to remove odor causing chemicals/toxic chemicals, smoke, fumes, bad breath etc. I.e., the devices hereof can reduce impacts from pathogens and/or odor, bad breath and perfume.

In one embodiment hereof, this product can be used by any two individuals who want to have a separation using a clear shield with side protectors and positive pressure air flow plus HEPA filters. In another embodiment hereof, this product can be used by any two individuals who want to have a separation using a clear shield with side protectors and negative pressure air flow plus HEPA filters. In another embodiment a unique design can be worn like a face shield with one or more light fans installed in the top of the unit and the device will be light and portable. The electricity to the one or more fans may be provided via a battery, e.g. often a rechargeable battery, though other plugged or corded power may be used instead. In another variation, the device comprises a power source, such as a battery, including, for example, a rechargeable battery such as a lithium battery. In another aspect, the power source is connected to a charger for charging the device. In another variation, the device may be equipped with one or more USB ports, such as a USB-A, Quick Charge USB or USB-C to charge the device.

In another implementation, the present application discloses a method for deflecting pathogens and may include:
a) a shield member; and, one or both:
b) a filtration sub-system; and/or
c) a positive pressure subsystem.

In another implementation, the present disclosure includes a method for deflecting pathogens and may include:
a) a shield member; and, one or more of:
b) a filtration sub-system; and/or
c) a positive pressure subsystem; and/or
d) a negative pressure subsystem.

In another implementation hereof, the device 550 may be wearable by an individual as shown in FIG. 14. The wearable device 550 may be curved to surround the head, face, and neck area of the individual. An adjustable band 552 may be used to secure the device 550 around the forehead region of an individual. The device 550 may have a top portion 554 that may include one or more optional sub-assembly or sub-assemblies 556 that include one or more fans or one more mini fans that provide an air pressure differential, typically positive pressure, at the top of the device. A grill or set of apertures 557 is shown as part of the top portion 554 to indicate air inlets for the positive pressure and/or filtration sub-assemblies. As positive pressure devices, the sub-assembly or sub-assemblies 556 may include one or more fans or one or more mini-fans; and, as filtration devices, these may include HEPA or other filtration arrangements. The number and/or size of fans can be varied relative to the practical application, personal usage implicating fewer and/or smaller fans (for e.g., practical weight and/or scale), larger scale operations indicating larger or more fans for/during operation. Only practicality limits how many or how large; whether no fans, one fan, a few fans, or a larger number of plural fans may be implemented. HEPA filter(s) may be used, and may be particularly applicable for bacteria and viruses and other filters might be used particularly for other desiderata; e.g., Carbon filter(s) may be used to reduce chemical odors such as odor or cosmetics, perfume, etc.

In another implementation of the developments hereof, negative pressure may be provided at the bottom of the device 500 to help draw air through the device in a consistent, constant, and/or unidirectional flow. This may be accomplished by an optional bottom portion 524 that may include one or more sub-assemblies 526 that include one or more fans or one or more mini-fans that provide negative pressure (or in some instances positive pressure) at the bottom of the device, as depicted in FIG. 15. It should be noted that many alternatives may be employed; e.g., in an alternative implementation, positive pressure may be provided from the bottom of the device 500 and negative pressure may be provided at the top of the device. In this way the device may create laminar flow along the device allowing smooth paths of air flow between the top and the bottom of the device. Or, pressure differential sub-assemblies 526 (negative or positive) might be used with prior described pressure differential sub-assemblies 506 (positive or negative) to increase laminar flow within the air space within the breath shield, or either of these may be used apart from each other. Laminar flow might be preferred (turbulent flow might be counter-active) to maximize air exchanges (volume of air completely moved through and replaced with a new volume of air), and though might be achievable with only one set of fans top or bottom, it might be that more efficiency or more laminar flow might be achievable with multiple placements of fans relative to the user air space. Also, the filtration part (or filter) of one or more sub-assemblies 506 might be disposed above in the top part 504 and yet may be used with negative pressure sub-assemblies 526 in another application.

In another aspect of the above device, a manual switch 530 for turning on and turning off the device may be a toggle or a push button installed on the front cover or on the side wall of the device. In another aspect of the above device, an LED 532 or status indicator may indicate whether the device is powered on and operating. In another aspect of the above device, an LED may indicate whether the filters are in need of replacement or whether fans may need maintenance or replacement (pressure differential monitoring and/or sensing, or time/period of use might be the activating factor). In some instances, one LED may be used to indicate the device is powered on/operating by providing a green (or other) color in a constant “on” state. In other instances, multiple LEDs may be used, for example, one to indicate status of the powered fans and another to indicate whether the filters or fans require replacement/maintenance. An LED indicator related to the status of the filters may have one or more colors associated with it, for example a green LED may be illuminated to show that the filters are on and functioning properly, an orange LED may be illuminated to demonstrate that the filters are nearing the end of their useful life, e.g. less than perhaps 24-48 hours e.g., of useful life left before requiring replacement, a red LED may be illuminated to indicate that one or more filters needs to be replaced. In another aspect of the above device, a speaker or audio component may be integrated with the device to provide an audible alarm to a user. The speaker or audio component may indicate that the filters need changing or another malfunction has been detected by the device.

FIG. 16 provides an example of electronic resources 600 that a device 500 hereof may have. FIG. 16 provides a non-limiting example of the schematic of the electronic resources 600, or in some instances a computer system or computing system, with which implementations hereof may be utilized. The computing system 600 may include a bus 610, at least one computer, processor, or microcontroller 612, memory 614, a power source 616, one or more sensors 618, one or more LEDs 620, and one or more fans, or fan arrays 622. In some implementations, the computing system 600 may include one or more wireless interface(s) 624, for example WIFI, Bluetooth Low Energy, Bluetooth, etc.). In some alternative implementations, the computing resources may include an audio emitters 626 (e.g. speaker/alarm) for providing audible signal or alert regarding an issue that may be encountered and detected by the device such as battery running low, one or more of the sensors 618 providing or detecting an abnormality or issue, or the fans or fan arrays 622 encountering an issue. In some implementations, the computing system may include one or more communication ports 628.

Processor(s) 612 can be any known processor, such as, but not limited to, an Intel® Atom® Processor x5 or x7 Cherry Trail, ARM Cortex-A72 or Cortex-A17, AMD® Ryzen™, or other similar type processors. Alternatively, such a processor 612 may be a Raspberry Pi 3B+, an Arduino, Orange Pi, or custom single board computer that may include one or more of the following: on board processing, storage, indicator light, micro SD slot, memory card slot, flash memory, WiFi, ethernet, USB power supply, RAM, and multi-pin connection points. The optional communication port(s) 628 can be any of an RS-232 for use with a modem based dialup connection, a 10/100 Ethernet port, a Universal Serial Bus (USB) port, or a Gigabit port using copper or fiber. Communication port(s) 628 may be chosen depending on a network such as a Local Area Network (LAN), Wide Area Network (WAN), or any network to which the computer system 600 connects or may be adapted to connect.

Memory 614 can be Random Access Memory (RAM), or any other dynamic storage device(s) commonly known in the art. Read only memory (not shown in FIG. 16) may also be included in the computing resources. The read only memory can be any static storage device(s) such as Programmable Read Only Memory (PROM) chips for storing static information such as instructions for processor 612. In some instances, the implementation may include, for example, 1 GB, 2 GB, 4 GB, of DDR3 RAM, or 1 GB, 2 GB, 4 GB, or 8 GB of DDR4-3200 single-channel RAM.

In some instances, mass storage (not shown in FIG. 16) may be connected to and used to store information and instructions. In other implementations, 64 GB or more, of soldered eMMC storage may be utilized.

Bus 610 may be used to communicatively couple the processor 612 with the other memory, storage, power, sensor, fans, speaker, and communication blocks.

In one example, the device hereof may include at least a processor 612 (for example Raspberry Pi 3B+, Arduino, or Orange Pi), memory 614, a source of power 616 (for example battery, wall plug, USB adapter, etc.) and one or more sensors 618.

The sensors 618 may include one or more of a pressure sensor, a differential air pressure sensor, a filter sensor, a humidity sensor, and a power sensor which may provide data for indication of operativeness, and/or health or life of filters or fans, perhaps needing attention, maintenance or replacement.

These implementations may be modified and adapted to and for different, substitute, or alternative components based on availability and for design for manufacture.

In another implementation of the developments hereof, one or more sensors may be provided and powered to detect airflow or to detect the status of filters. For example, a differential pressure sensor may be utilized to measure the flow rate of air space near or adjacent the user and the primary shield portion 501. In one aspect a gauge pressure sensor may be exposed to ambient air and another sensor is exposed to air within close proximity to the stream of air near the primary shield portion. Health or need for replacement of filters may be indicated by such sensors; or, health or repair or maintenance or replacement of a fan or fans might be indicated also. Timing alarms could be used for similar health or repair or replacement of such parts as well or instead.

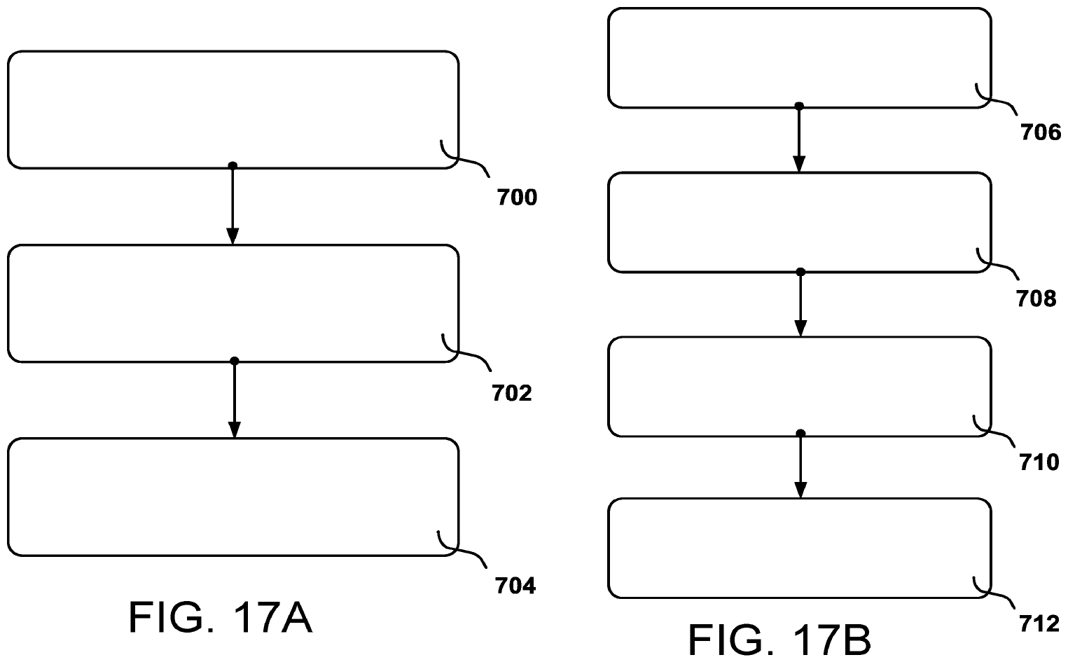

FIG. 17A provides a flow chart showing an exemplar methodology for operating a device hereof. The method may include powering on the device 700 including one or more fans or fan and/or filter arrays, sensing and/or detecting 702 one or more of the flow of air, the quality of the air (via the filter sensor), and/or the humidity of the air, at the device using the one or more pressure sensors or differential air pressure sensor, the filter sensor, and/or humidity sensor, and providing an alarm or alerting 704 if the air flow is too low or of other conditions observable using the one or more sensors.

FIG. 17B provides a flow chart showing another exemplar methodology for operating a device hereof. The method may include positioning the device or shield 706, which may include one or more of inserting the slit lamp device through the aperture on the primary shield portion (see FIG. 1 inter alia) or positioning on a user (as in FIG. 14), providing power or powering on the device 708 including one or more fan or fan arrays, sensing and/or detecting 710 one or more of the flow of air, the quality of the air (via the filter sensor), and/or the humidity of the air, at the device using the one or more pressure sensors or differential air pressure sensor, the filter sensor, and/or humidity sensor, and providing an alarm or alerting 712 if the air flow is too low or of other conditions observable using the one or more sensors.

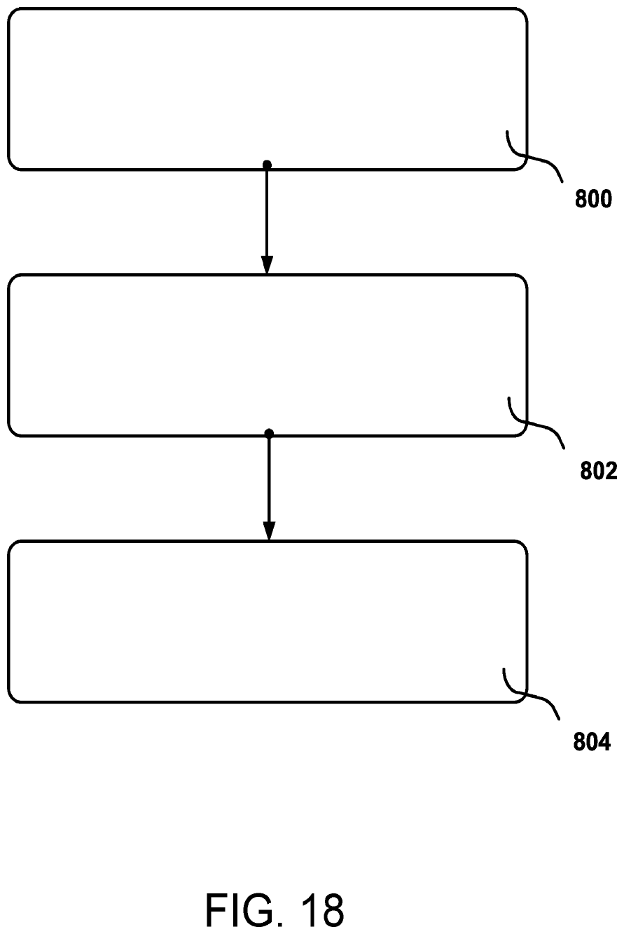

FIG. 18 provides a flow chart showing another exemplar methodology for operating a device hereof. The method may including positioning 800 the device relative to the user, establishing 802 a pressure differential in the breathing space/area, and cleaning air by forcing 804 the air through the filter, often but limited to a HEPA filter. Various alternatives and/or additions may be made with these methods, whether by defining the type of pressure differential (positive and/or negative) or defining a type of filter or filtration, or defining the use disposition (on a device or platform, or on a user him/herself), e.g.

Various aspects described herein may be embodied as an apparatus, a device or a method. Accordingly, those aspects may take the form of an entirely hardware and electronic implementation. Aspects of the disclosure have been described in terms of illustrative implementations and variations thereof. Numerous other implementations, modifications and variations within the scope and spirit of the appended claims will be ascertained to persons of ordinary skill in the art from a review of this disclosure. For example, one of ordinary skill in the art will appreciate that the steps illustrated in the illustrative implementations and figures may be performed in other than the recited order, and that one or more steps illustrated may be optional in accordance with aspects of the disclosure.

What is claimed:

1. A device, system and/or method as described and disclosed herein.

2. A device, system and/or method for pathogen deflection including:
a) a shield member; and, one or both:
b) a filtration sub-system; and/or
c) a pressure differential subsystem.

3. A device, system and/or method according to either of claim 1 or 2 for pathogen deflection, the shield member including a primary shield member.

4. A device, system and/or method according to any of claim 1 or 2 or 3 for pathogen deflection, the shield member including one or more of a first side portion, a second side portion and a top portion.

5. A device, system and/or method according to any of claims 1-4 for pathogen deflection, the shield member including a slot or aperture for reception of a portion of a slit lamp device or like device.

6. A device, system and/or method according to claims 1-5 for pathogen deflection, the shield member including a top portion adjacent the main front portion.

7. A device, system and/or method according to claims 4, 5 or 6 for pathogen deflection, the shield member including a grill or set of apertures attached to or as part of the top portion to indicate air inlets or exits.

8. A device, system and/or method according to any of claims 1-7 for pathogen deflection, the inlets or exits for the pressure differential and/or filtration sub-assemblies.

9. A device, system and/or method according to any of claims 1-8 for pathogen deflection, the shield member having a top member including one or more wells for the filtration and/or pressure differential sub-assemblies.

10. A device, system and/or method according to any of claims 1-9 for pathogen deflection, the top member including a lip member which may be attached to and/or form a part of the top member to assist in directing air pressure differential flow and/or further assist in deflection.

11. A device, system and/or method according to any of claims 1-9 for pathogen deflection, the shield member being cut, molded or otherwise formed as a relatively single part that may initially be flat and then bent to take the forms hereof.

12. A device, system and/or method according to any of claims 1-11 the filtration and/or pressure differential sub-assemblies may be or may include:
positive or negative pressure and/or filtration sub-assemblies which may be:
as positive or negative pressure devices, these may include one or more fans or one or more mini-fans; and,
as filtration devices, these may include HEPA or charcoal other filtration arrangements.

13. A method for deflecting pathogens including:
a) a shield member; and, one or both:
b) a filtration sub-system; and/or
c) a pressure differential subsystem.

14. A device, system and/or method according to any of claims 1-13 the filtration and/or pressure differential sub-assemblies may be or may include:
positive pressure and/or filtration sub-assemblies which may be:
as positive pressure devices, these may include one or more fans or one or more mini-fans; and,
as filtration devices, these may include HEPA or charcoal other filtration arrangements;
negative pressure sub-assemblies which may include one or more fans or one or more mini-fans.

15. A device, system and/or method according to any of claims 1-14 having one or more filter sensors.

16. A device, system and/or method according to any of claims 1-15 having one or more air flow sensors.

17. A device, system and/or method according to any of claims 1-16 having one or more LEDs to indicate status of power, filter health, and/or air flow.

18. A device, system and/or method according to any of claims 1-17 for pathogen deflection, the shield member including a slot or aperture for reception of a portion of a slit lamp device or like device.

19. A device, system and/or method according to claim 18 further including a gasket or baffle occupying a substantial portion of the aperture.

20. A device, system and/or method according to any of claim 19 for pathogen deflection, the gasket or baffle being made of rubber, vinyl, latex, plastic or other partially deformable material.

21. A device, system and/or method according to any of claims 1-20 further comprising a stand.

22. A method for cleaning air in an air space in or adjacent a breath shield, comprising:
positioning the device relative to the user;
establishing a pressure differential in the breathing space/area; and
cleaning air by forcing the air through a filter.

23. A method according to claim 22 the positioning including positioning the breath shield on a surface or a device.

24. A method according to claim 23 the positioning including positioning the breath shield on or in operative relation to a medical device.

25. A method according to claim 25 the positioning including positioning the breath shield on an ophthalmology device.

26. A method according to any of claims 22-25 the establishing including providing a positive pressure for air flow.

27. A method according to any of claims 22-25 the establishing including providing a negative pressure for air flow.

28. A method according to any of claims 22-25 the establishing including providing the positive and negative pressure for air flow.

29. A method according to any of claims 22-28 the establishing including providing the laminar air flow.

30. A method according to any of claims 22-29 the cleaning including providing pathogen reduction.

31. A method according to any of claims 22-30 the cleaning including a HEPA filter and forcing air through the HEPA filter.

32. A method according to any of claims 22-31 the cleaning including a carbon filter and forcing air through the carbon filter.

33. A method for cleaning air comprising:
powering on a device including one or more fans or filter arrays,
sensing and/or detecting one or more of the flow of air, the quality of the air, and/or the humidity of the air, at the device and
providing an alarm or alerting if the air flow is too low or of other conditions observable using the one or more sensors.

34. A method according to claim 33 including the one or more pressure sensors or differential air pressure sensor, the filter sensor, and/or a humidity sensor.

35. A method for operating a device for cleaning air in an airspace, the device including one or more fan and/or filter arrays; comprising:
positioning the device,
providing power or powering on the device,
sensing and/or detecting one or more of the flow of air, the quality of the air, and/or the humidity of the air, at the device, and
providing an alarm or alerting if the air flow is too low or of other conditions observable using the one or more sensors.

36. A method according to claim 35 including the one or more pressure sensors or differential air pressure sensor, the filter sensor, and/or a humidity sensor.
